(19) United States
(12) Patent Application Publication
(10) Pub. No.: US 2013/0196315 A1
(43) Pub. Date: Aug. 1, 2013

(54) DEVICE FOR CELL CULTURE AND ANALYSIS

(71) Applicants: Marco Chilosi, Verona (IT); Mauro Krampera, Verona (IT); Mario Ricciardi, Verona (IT)

(72) Inventors: Marco Chilosi, Verona (IT); Mauro Krampera, Verona (IT); Mario Ricciardi, Verona (IT)

(73) Assignee: UNIVERSITA' DEGLI STUDI DI VERONA, Verona (IT)

(21) Appl. No.: 13/580,736

(22) PCT Filed: Feb. 23, 2011
(86) PCT No.: PCT/IB11/00365

§ 371 Date: Nov. 1, 2012

(30) Foreign Application Priority Data
Feb. 23, 2010 (IT) TO2010U000026

(51) Int. Cl. C12Q 1/04

(52) U.S. Cl. CPC C12Q 1/04
USPC 435/6.11; 435/288.1; 435/7.1

(57) ABSTRACT

The present invention relates to a device for cell culture and analysis, comprising a first containing element, a second containing element sealingly couplable to one another to define a culture chamber and a flexible film housable in said culture chamber and available alternatively on one of said first element or second element. The flexible film can be made of a transparent material selected from the group consisting of polycarbonate, polystyrene and polyethylene. A method for cell analysis is also provided.

### TECHNICAL FIELD

The present invention relates to a device for cell culture and analysis.

### BACKGROUND ART

It is known to use flasks for cell culture to allow cell growth in vitro.

In particular, these flasks have a substantially parallelepipedal shape and are provided, on a face having a smaller surface with a tubular neck defining an opening adapted to be sealingly closed by a cap.

The flask internally defines a parallelepipedal culture chamber. A culture medium is placed in the chamber by introduction through the neck; the cells are then seeded on this culture medium.

In particular, the culture medium is normally placed on an inner face of the culture chamber that has a larger surface.

To promote cell adhesion, the inner face of the culture chamber is treated, by means of known methods, to produce an electrostatically positively charged or negatively charged surface.

After seeding the cells, the sealed flask is maintained in a controlled temperature environment to promote cell growth.

In many cases, once a cell population is obtained, the presence of a specific group of proteins must be determined on the cell in order to characterise the cell type or determine the biological effect thereof.

Protein expression is nowadays normally tested by immunofluorescence or immunohistochemistry. However, these methods are not suitable for the screening of many proteins in a high number of samples (high-throughput screening, HTP) both for the large amount of cells required, and due to the time- and material-consuming and expensive procedures. For example, up to now, the number of minimum starting cells for each single support (glass or flask) is about 50.000 units; the markers that may be characterised on the same support are normally less than 5 and the times are long, as the protocols are not automated.

The need is therefore felt in the field for a device that allows to apply immunohistochemistry and immunofluorescence techniques even to high-throughput screening.

In particular, the need is felt in the field to provide a device that makes lab tests simpler for characterising primary cell lines and stem cells after protocols for differentiation of neoplastic populations, of immunophenotypic alterations, as well as for identifying new drugs and evaluating their biological effect.

### DISCLOSURE OF INVENTION

It is the object of the present invention to provide a cell growth device which allows to satisfy in a simple and cost-effective manner one of the above said needs.

This object is achieved by the present invention, as it relates to a cell culture and cell analysis device according to claim 1 and a method for cell analysis according to claim 11.

### BRIEF DESCRIPTION OF THE DRAWINGS

A preferred embodiment is hereinafter disclosed for a better understanding of the present invention, by mere way of non-limitative example and with reference to the accompanying drawings, in which:

FIG. 1 is a perspective view that shows a cell culture device according to a first embodiment of the invention;

FIG. 2 is an exploded perspective view of the cell culture device of FIG. 1;

FIG. 3 is a perspective view that shows the cell culture device according to a second embodiment of the invention;

FIG. 4 shows with a greater detail the flexible film shown in FIG. 2;

FIG. 5 shows a first method for the use of the cell culture device according to the present invention; and

FIG. 6 shows an alternative method with respect to that shown in FIG. 5 for the use of the cell culture device according to the present invention.

### BEST MODE FOR CARRYING OUT THE INVENTION

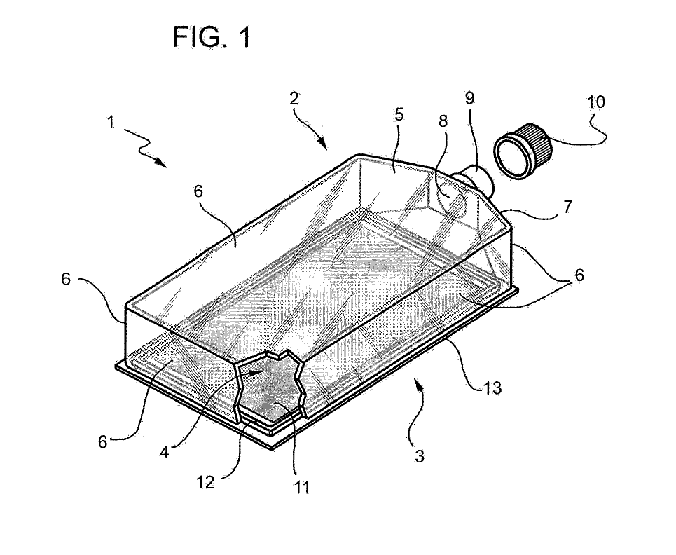

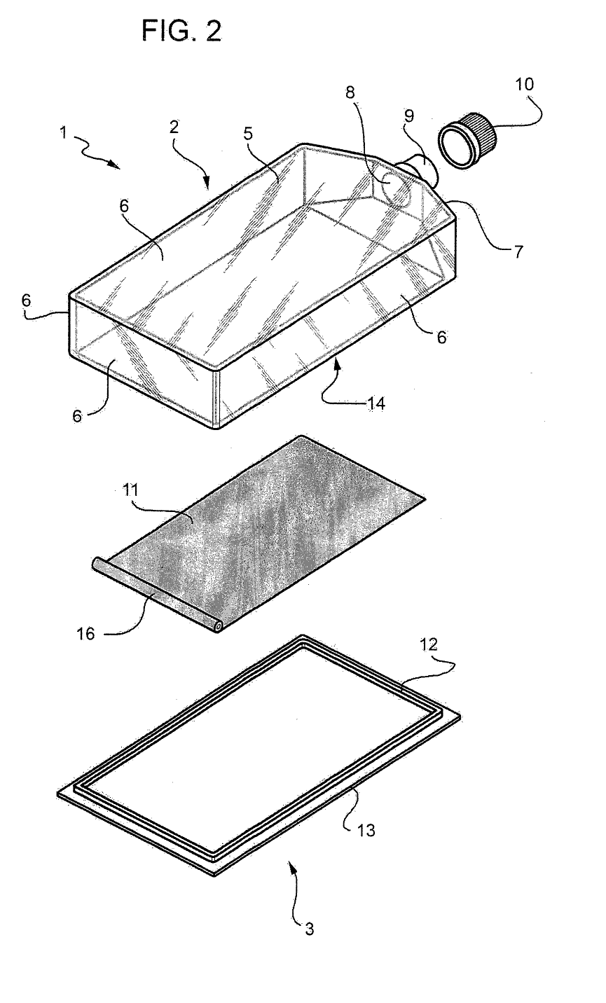

FIGS. 1 and 2 indicate with numeral 1 as a whole a cell culture and analysis device. In the case shown, the cell growth device is a flask for cell culture.

Flask 1 comprises a first containing element 2 and a second containing element 3 sealingly couplable to one another to define a culture chamber 4 (in FIG. 1 elements 2 and 3 are represented coupled while in FIG. 2 elements 2 and 3 are represented as separate).

First element 2 comprises a bottom wall 5 having a substantially rectangular flat shape integral with side walls 6 which extend along peripheral edges of the bottom wall 5 and are transversal to the bottom wall 5. In the figures, side walls 6 are shown arranged perpendicularly with respect to bottom wall 5.

In particular, a cylindrical tubular appendix 9 extends integrally from a central portion 7 of a side wall 6 having, in the embodiment shown, a smaller surface.

Tubular appendix 9 extends outwards from device 1 and defines a circular opening 8 to allow the communication with the outside of culture chamber 4, for example to introduce/replace the culture medium.

Tubular appendix 9 may be closed by removable sealing means 10, for example a cap which is screwed/unscrewed on the end portion of tubular appendix 9 which for this purpose is provided with helicoidal ribs.

Second element 3 has a substantially flat rectangular configuration and is provided with peripheral edges 13 adapted to sealingly couple with corresponding peripheral edges 14 of side walls 6.

In use, second element 3 forms a wall of flask 1 arranged spatially in a facing position opposite to bottom wall 5.

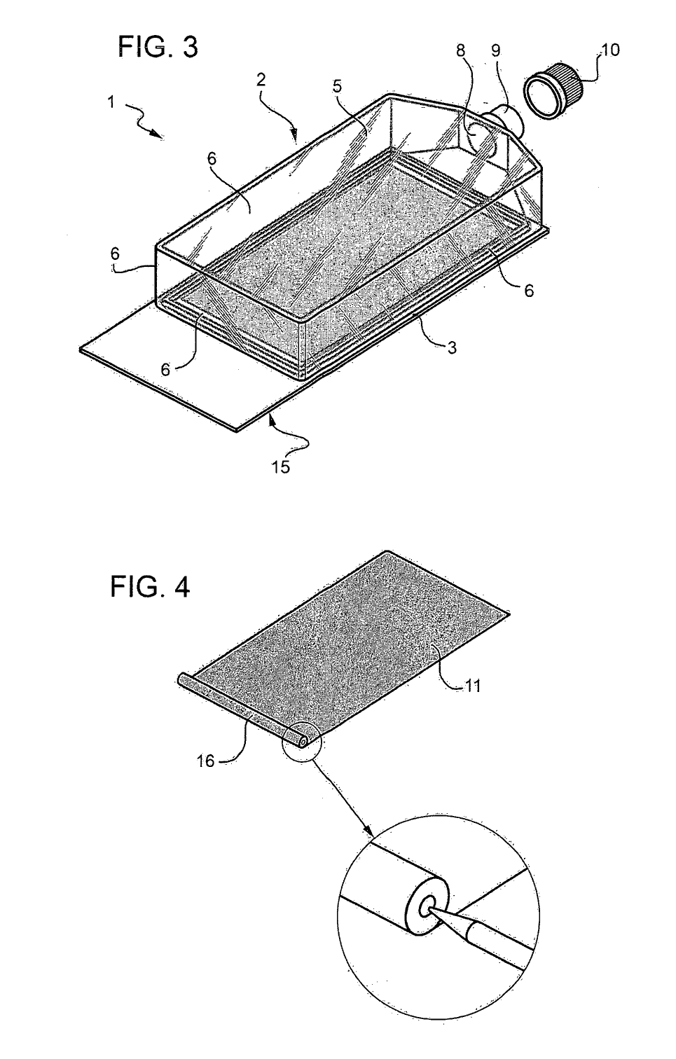

Second rectangular flat element 3 may have dimensions substantially corresponding to that of bottom wall 5 as shown in FIG. 1 or 2 or may have at least one dimension greater than the corresponding dimension of bottom wall 5. In FIG. 3, for example, the larger side of second rectangular element 3 has a longer length with respect to the length of the corresponding side of bottom wall 5 so as to provide a rectangular flat flange 15 that projects with respect to first element 2.

Advantageously, the embodiment shown in FIG. 3 makes the assembly/disassembly of device 1 easier also for more conveniently handling the cell sample. Furthermore, flange 15 can carry identification elements, for example a label, to simplify the identification of the sample.

First element 2 and second element 3 can be sealingly coupled by means of snap coupling means 12, for example a peripheral groove made on the inner surface of second element 3 adapted to house at least the end portion of side walls 6 of first element 2, or alternatively a peripheral projection on the inner surface of second element 3 that abuts with side walls 6 of first element 2. Alternatively the sealing coupling between first element 2 and second element 3 can be made by ultrasound welding and/or glues. Coupling means 12 are such as to maintain the impermeability of the flask to the outside environment in use, although they can be manually forced to allow the operation of first element 2 and of second element 3 and therefore the opening of flask 1 making the rinsing and possibly a second use easier.

First element 2 and second element 3 are made of a rigid and transparent material such as for example polystyrene, polycarbonate or glass.

Flask 1 further comprises a flexible film 11 housable within culture chamber 4 and available on the inner surface of second rectangular flat element 3 or alternatively on the inner surface of bottom wall 5 of first element 2. In the embodiment of the figures, film 11 has a rectangular perimeter.

This flexible film 11 is treated by means of known methods to obtain an electrostatically positive or negative charged surface to provide cell growth. It is made of a transparent material, preferably selected from the group consisting of polycarbonate, polystyrene and polyethylene, to allow its use for example in microscope analysis. Furthermore, the flexibility of the material advantageously allows to roll and fold film 11 for subsequent immunofluorescence or immunohistochemistry analysis. As shown in FIG. 4a, flexible film 11 may also be provided with winding means 16 to allow the winding of film 11 on itself. In particular, winding means 16 consist of a cylindrical thickening that can have a hollow section made of the same material of film 11 (or of another material compatible with microtome and/or cryostat cutting) within which a stick adapted to aid the winding of the film on itself can for example be inserted. The cylindrical thickening may alternatively be provided at each of its ends with an end portion 21 projecting with respect to flexible film 11 and mobile between a first position folded over the thickening and a second extended position in which it aids the winding of flexible film 11 on itself, as shown in FIG. 4b.

In use, flexible film 11 is fixed on second element 3, for example by means of an adhesive. Subsequently, second element 3 is coupled to first element 2 so as to arrange flexible film 11 within chamber 4 and allow the laying and cell growth on flexible film 11.

Alternatively, flexible film 11 can be fixed interposing it between first element 2 and second element 3, coupling means 12 of which retain a peripheral portion of flexible film 11.

Advantageously, after the cell culture cycle has been completed or the fresh sample has been laid, flexible film 11 carrying the cells is easily and rapidly removed from culture chamber 4 as the uncoupling of elements 2, 3 is simple and fast and uncoupled second element 3 forms a support for flexible film 11.

Thereby, flexible film 11 can be subjected directly to biological analyses such as immunohistochemistry and immunofluorescence. Flexible film 11 is also compatible with other standard hybridisation techniques such as FISH and in situ hybridisation. Finally, the cells on flexible film 11 can be fixed with formaldehyde or paraformaldehyde, can be included in paraffin and analysed directly using HTS systems to identify the expression profile of the cells.

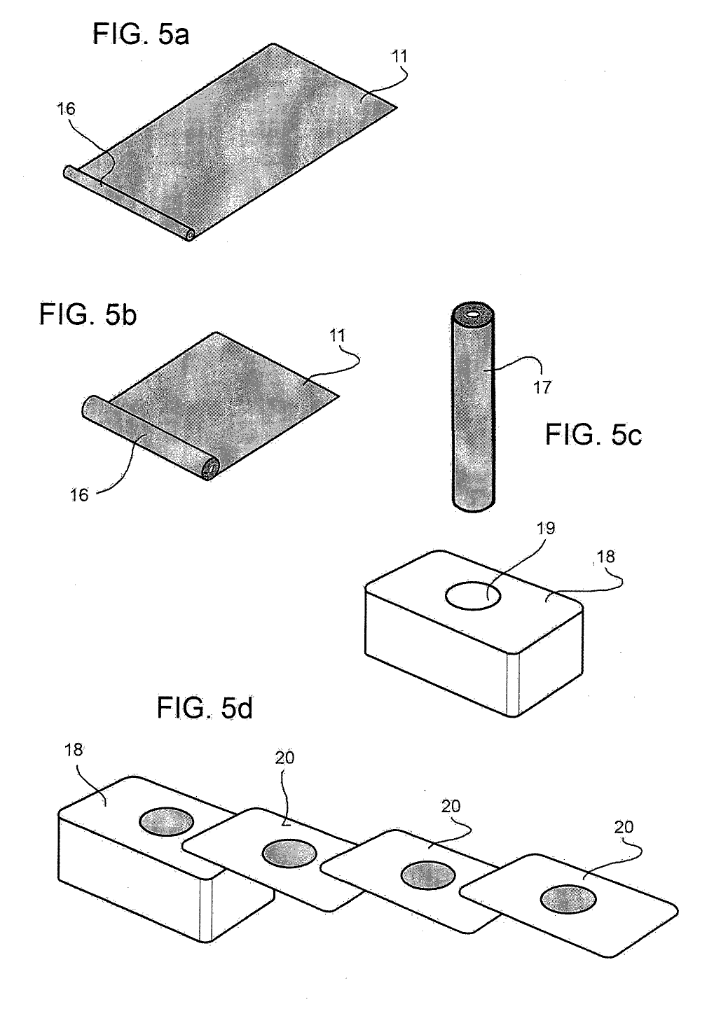

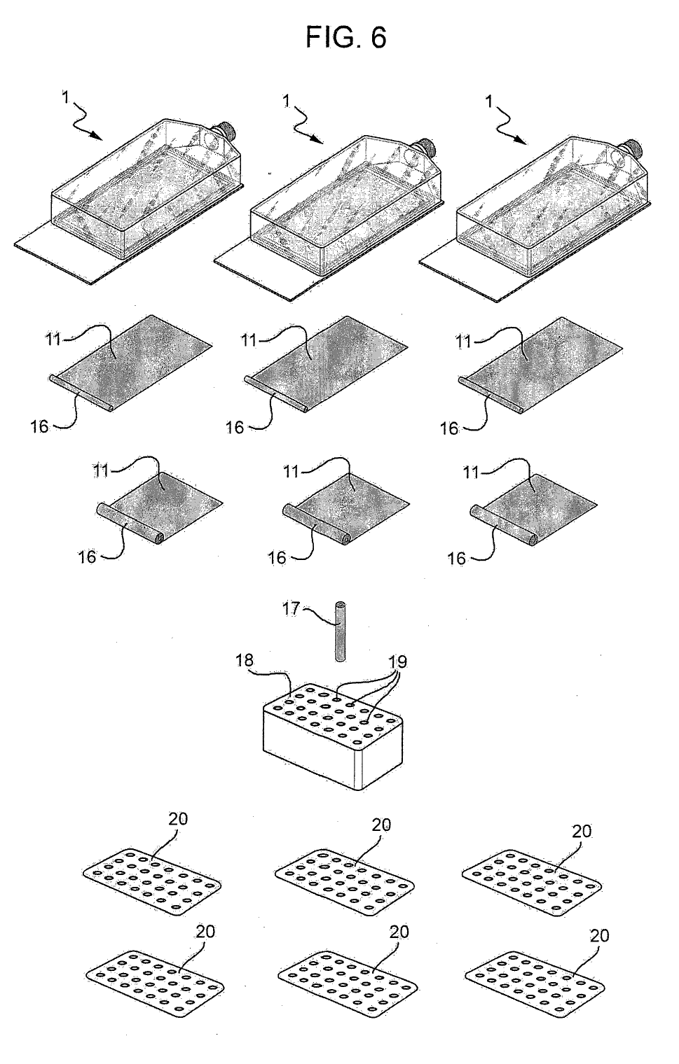

FIGS. 5 and 6 show two possible embodiments of the method according to the invention, in particular, they show two methods for using film 11 once removed from flask 1, in order to characterise the protein alterations of the cells grown in the flask.

In FIG. 5, film 11 carrying the adhering cells is fixed by known methods. As shown in FIG. 5b, film 11 is then wound on itself by means of winding means 16 to obtain a cylinder 17 (FIG. 5c). Cylinder 17 obtained thereby, is inserted vertically within a paraffin block 18 in which a cavity 19 has been created having dimensions substantially corresponding to those of cylinder 17. Paraffin block 18 including cylinder 17 can be treated the same way as a biological tissue and therefore can be cut in thin slices 20, as shown in FIG. 5d. The characterisation by IHC or IF on these slices 20 can be performed for example manually or by automatic robot processing. Several slices 20 can also be generated from each single cylinder 17. A single IHC or IF analysis can be performed on each slice.

Similarly to what has been disclosed for FIG. 5, FIG. 6 shows a possible use of the invention to carry out the comparison of a same treatment on different cells or alternatively evaluate the effect of different treatments on a same cell sample. Similarly to what has been disclosed for FIG. 5, cylinders 17 obtained from different flasks 1 are introduced in a single paraffin block 18 in which several cavities 19 have been created corresponding to the number of cylinders 17 to be analysed and having dimensions substantially corresponding thereto. Cavities 19 are preferably arranged according to a matrix structure. Figure highlights how multiple cylinders 17 deriving from different flasks 1 can be included in a single paraffin block 18 in order to optimize at best times and costs of the IHC and/or IF reactions.

Finally, it is clear that modifications and variants not departing from the scope of protection of the independent claims can be made to the disclosed and shown system.

1. A device (1) for cell culture and analysis, comprising a first containing element (2), a second containing element (3) sealingly couplable to one another to define a culture chamber (4) and a flexible film (11) housable in said culture chamber (4) and available alternatively on one of said first element (2) or second element (3).

2. The device according to claim 1, wherein said flexible film (11) is made of a material selected from the group consisting of polycarbonate, polystyrene and polyethylene.

3. The device according to claim 1 or 2, wherein said first containing element (2) and said second containing element (3) are made of transparent material.

4. The device according to claim 3, wherein said transparent material is selected from the group consisting of polystyrene, polycarbonate and glass.

5. The device according to claim 1, wherein said first containing element (2) comprises a substantially flat bottom wall (5) integral with side walls (6) which extend along peripheral edges of said bottom wall (5) and are transversal to said bottom wall (5);
said second element (3) comprising a substantially flat wall provided with peripheral edges (13) adapted to sealingly couple with corresponding peripheral edges (14) of said side walls (6).

6. The device according to claim 1, wherein at least one of said first containing element (2) and said second containing element (3) is provided with a tubular appendix (9) which extends outside the device and defines an opening (8) communicating with said culture chamber (4); said opening (8) being closable by removable sealing means (10).

7. The device according to claim 1, wherein said second containing element (3) defines at least one flat portion on which said flexible film (11) is arranged.

8. The device according to claim 1, characterised in that said second containing element (3) has an essentially flat configuration and has at least one dimension larger than the corresponding dimension of said bottom wall (5) so as to form a flange (15).

9. The device according to claim 1 or 2, wherein said flexible film (11) is provided with winding means (16) to allow the winding of said flexible film (11) on itself.

10. The device according to any of the preceding claims, wherein said first element (2) and said second element (3) are sealingly coupled by snap coupling means (12).

11. A method for cell analysis comprising the steps of:
a) culturing the cells to be analysed on a flexible film (11);
b) winding said flexible film (11) by means of winding means (16) to obtain a cylinder (17);
c) providing a support (18) provided with at least one cavity (19) having dimensions substantially corresponding to those of the cylinder (17);
d) inserting at least one of said cylinders (17) in a respective cavity (19);
e) sectioning said support (18) along a direction transversal to the axis of said cavity (19) so as to obtain at least one section (20) of said support (18) incorporating a section of said cylinder (17);
f) subjecting said section (20) to said cell analysis.

12. The method according to claim 11, wherein said support (18) is provided with a plurality of cavities (19) adapted to house respective cylinders (17).

13. The method according to claim 12, wherein said cavities (19) are arranged according to a matrix structure.
